(19) United States
(12) Patent Application Publication
(10) Pub. No.: US 2012/0157731 A1
(43) Pub. Date: Jun. 21, 2012

(54) Multistage Prereforming

(75) Inventor: Bhadra S. Grover, Sugar Land, TX (US)

(73) Assignee: L'Air Liquide Societe Anonyme Pour L'Etude Et L' Exploitation Des Procedes Georges Claude, Paris (FR)

(21) Appl. No.: 12/970,041

(22) Filed: Dec. 16, 2010

(51) Int. Cl. C07C 4/06

(52) U.S. Cl. 585/310

(57) ABSTRACT

A pre-reforming process is provided. This process includes heating a first stream containing heavy hydrocarbons to a first temperature, then introducing the heated first stream into a first pre-reforming chamber, thereby producing a first pre-reformed stream. This process also includes heating the first pre-reformed stream to a second temperature, then introducing the heated first pre-reformed stream into a second pre-reforming chamber, thereby producing a second pre-reformed stream. This process also includes heating the second pre-reformed stream to a third temperature, then introducing the heated second pre-reformed stream into a third pre-reforming chamber, thereby producing a third pre-reformed stream.

### BACKGROUND

Prereforming of hydrocarbons upstream of the SMR or ATR is a well known process. It converts heavier hydrocarbons (ethane and heavier) to methane. It may also convert some of the methane to hydrogen, CO, and CO2, depending upon the chemical equilibrium under the given conditions.

Prereformer utilizes waste heat in the flue gas or process stream, which otherwise may be utilized in raising steam. Utilization of high level heat (at about 1600° F. to about 900° F.) is thermodynamically more efficient when used for prereforming than for raising steam with boiling temperature of about 400° F. to 600° F. Disposal of excess steam is a problem in many plants. Typically the feed (hydrocarbon and steam mixture) to the prereformer is preheated in the range of 850° F. to 1000° F. before contacting with a catalytic bed in an adiabatic reactor. The reactants come to a chemical equilibrium. The extent of conversion of methane to H2/CO/CO2 is a function of the reaction temperature, higher temperature favoring the conversion.

The inlet temperature of the feed to prereformer is limited by its potential to crack hydrocarbons and deposit carbon on the catalyst and the preheat coils. Heavier the feedstock, lower is the potential cracking temperature. For example, the feed temperature for typical light natural gas is limited to about 1000° F., while feed temperature for naphtha feed is limited to 850° F. The amount of waste-heat utilization for prereforming depends on the preheat temperature of feed mixture. There is a need for a process that can utilize larger amounts of waste heat for prereforming.

### SUMMARY

In one embodiment of the present invention, a pre-reforming process is provided. This process includes heating a first stream containing heavy hydrocarbons to a first temperature, then introducing the heated first stream into a first pre-reforming chamber, thereby producing a first pre-reformed stream. This process also includes heating the first pre-reformed stream to a second temperature, then introducing the heated first pre-reformed stream into a second pre-reforming chamber, thereby producing a second pre-reformed stream. This process also includes heating the second pre-reformed stream to a third temperature, then introducing the heated second pre-reformed stream into a third pre-reforming chamber, thereby producing a third pre-reformed stream.

### BRIEF DESCRIPTION OF THE FIGURES

FIG. 1 illustrates a representation of one embodiment of the present invention.

FIG. 2 illustrates a representation of another embodiment of the present invention.

### DESCRIPTION OF PREFERRED EMBODIMENTS

Illustrative embodiments of the invention are described below. While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

A method to increase the extent of prereforming and higher utilization of waste heat for prereforming is herein proposed. The reaction products from the first prereformer reactor is heated to a higher temperature by exchanging heat with flue gas or process gas, and sent to a second adiabatic catalytic reactor. Since most of the heavy hydrocarbons have already been converted to methane in the first prereformer, the reactants to second prereformer can safely be heated to about 1150° F. to 1200° F. without risk of cracking and carbon formation. The reaction in the second prereformer is mostly conversion of methane to H2 and CO, which is endothermic. The reaction products will be close to chemical equilibrium at a temperature that may be 100 to 150° F. lower than the reactants inlet temperature. The reaction products from the second prereformer reactor can be heated once again to about 1200° F., utilizing some more waste heat, and fed to a third adiabatic catalytic prereformer reactor. This can be repeated in a fourth and fifth reactor. However, the benefit of adding beyond third reactor is greatly diminished.

Presence of H2 in the reactants (methane, steam mixture) increases the cracking temperature. Concentration of H2 increases at each stage of prereforming, allowing its products to be heated to a higher temperature for next stage of prereforming.

The first stage of prereforming normally provides 8-10% of hydrocarbon conversion. Additional two stages of prereforming as described above can increase the hydrocarbon conversion to about 20-25%. Correspondingly, it also reduces amount of steam that need to be raised to utilize the waste heat.

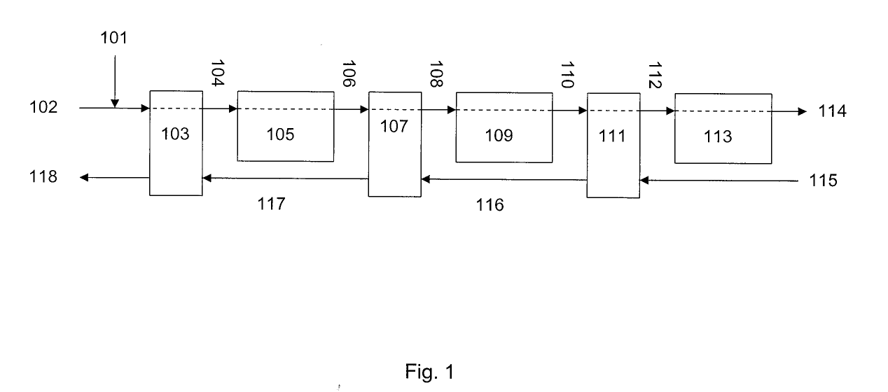

Turning to FIG. 1, a pre-reforming process is disclosed. A first stream containing heavy hydrocarbons 101 and steam 102 is heated to a first temperature in first vessel 103, by indirect heat exchange with hot gas stream 117, thereby producing first reformer inlet stream 104. First reformer inlet stream 104 is then introduced into first pre-reforming chamber 105, thereby producing first pre-reformed stream 106.

First pre-reformed stream 106 is heated to a second temperature in second vessel 107, by indirect heat exchange with hot gas stream 116, thereby producing second reformer inlet stream 108. Second reformer inlet stream 108 is then introduced into second pre-reforming chamber 109, thereby producing second pre-reformed stream 110.

Second pre-reformed stream 110 is heated to a third temperature in third vessel 111, by indirect heat exchange with hot gas stream 115, thereby producing third reformer inlet stream 112. Third reformer inlet stream 112 is then introduced into third pre-reforming chamber 113, thereby producing third pre-reformed stream 114. Third pre-reformed stream 114 may then be heated once again in a fourth heat exchanger (not shown) prior to usage downstream. Note in one embodiment, hot gas stream 117, hot gas stream 116, and hot gas stream 115 may come from different sources (not shown).

The second temperature may be greater than said first temperature. The third temperature may be greater than said second temperature. The indirect heat exchange may be with a flue gas from an SMR furnace. The indirect heat exchanger may be with one or more process streams.

The first temperature may be less than 1100 F, preferably less than 1020 F. The second temperature may be less than 1200 F, preferably less than 1150 F. The third temperature may be between about 1200 F and about 1300 F.

The amount of steam mixed with hydrocarbons depends on the catalyst, and the type of hydrocarbon feedstock. The skilled artisan will be able to select the proper amount of steam for any application without undue experimentation.

At least one of the pre-reforming chambers may contain Ni catalyst or precious metal catalyst. At least one of the pre-reforming chamber may contain Ni catalyst and at least one other pre-reforming chamber may contain precious metal catalyst. The precious metal catalyst may be selected from the group consisting of Pt, Pd, and Ru.

Each pre-reforming chamber may be a stand alone reactor. At least two pre-reforming chambers may be contained in a single vessel. The first pre-reforming chamber may have a first space velocity, the second pre-reforming chamber may have a second space velocity, and the third pre-reforming chamber may have a third space velocity, where the first space velocity is lower than said second space velocity or said third space velocity.

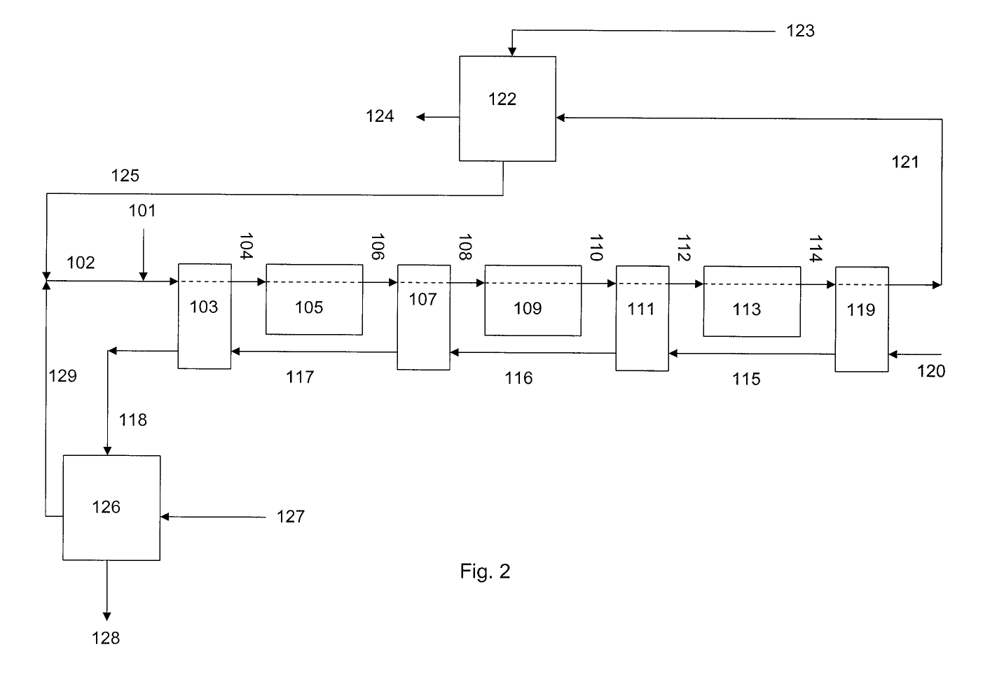

Turning to FIG. 2, In the interest of simplicity, the same element numbering scheme is used in both figures. A first stream containing heavy hydrocarbons 101 and steam 102 is heated to a first temperature in first vessel 103, by indirect heat exchange with hot gas stream 117, thereby producing first reformer inlet stream 104, and hot gas stream 118. First reformer inlet stream 104 is then introduced into first pre-reforming chamber 105, thereby producing first pre-reformed stream 106.

First pre-reformed stream 106 is heated to a second temperature in second vessel 107, by indirect heat exchange with hot gas stream 116, thereby producing second reformer inlet stream 108. Second reformer inlet stream 108 is then introduced into second pre-reforming chamber 109, thereby producing second pre-reformed stream 110.

Second pre-reformed stream 110 is heated to a third temperature in third vessel 111, by indirect heat exchange with hot gas stream 115, thereby producing third reformer inlet stream 112. Third reformer inlet stream 112 is then introduced into third pre-reforming chamber 113, thereby producing third pre-reformed stream 114. Third pre-reformed stream 114 may then be heated once again in a fourth heat exchanger (not shown) prior to usage downstream. Note in one embodiment, hot gas stream 117, hot gas stream 116, and hot gas stream 115 may come from different sources (not shown).

Third pre-reformed stream 114 is then introduced into reformer 119 as feed. The reformer 119 may be a steam methane reformer or an autothermal reformer. The inter-stage heating may be performed by the flue gas of a process heater for preheating other process streams, where the inter-stage heating may be an integral part of the autothermal reforming process.

Reformer 119 requires a fuel inlet 120, produces a first reformed gas stream 121, and hot gas stream 115. First reformed gas stream 121 may be introduced into a first waste heat steam generator 122, which requires a boiler feed water inlet stream 123, and produces a flue gas stream 124, and a first steam stream 125.

Hot gas stream 118 may be introduced into a second waste heat steam generator 126 (or it could be used for other services such as combustion air preheat, process gas preheat, etc.), which requires a boiler feed water inlet stream 127, and produces a flue gas stream 128, and second steam stream 129. First steam stream 125 and second steam stream 125, may be combined to form steam 102.

What is claimed is:

1: A pre-reforming process comprising,
heating a first stream containing heavy hydrocarbons to a first temperature, then introducing said heated first stream into a first pre-reforming chamber, thereby producing a first pre-reformed stream;
heating said first pre-reformed stream to a second temperature, then introducing said heated first pre-reformed stream into a second pre-reforming chamber, thereby producing a second pre-reformed stream; and
heating said second pre-reformed stream to a third temperature, then introducing said heated second pre-reformed stream into a third pre-reforming chamber, thereby producing a third pre-reformed stream.

2: The process of claim 1, wherein said second temperature is greater than said first temperature.

3: The process of claim 1, wherein said third temperature is greater than said second temperature.

4: The process of claim 1, wherein said heating is by indirect heat exchange with a flue gas from an SMR furnace.

5: The process of claim 1, wherein said heating is by indirect heat exchange with one or more process streams.

6: The process of claim 1, wherein said first temperature is less than 1100 F.

7: The process of claim 6, wherein said first temperature is less than 1020 F.

8: The process of claim 1, wherein said second temperature is less than 1200 F.

9: The process of claim 8, wherein said second temperature is less than 1150 F.

10: The process of claim 1, wherein said third temperature is between about 1200 F and about 1300 F.

11: The process of claim 1, wherein pre-reforming chambers contain Ni catalyst.

12: The process of claim 1, wherein pre-reforming chambers contain precious metal catalyst.

13: The process of claim 12, wherein said precious metal catalyst is selected from the group consisting of Pt, Pd, and Ru.

14: The process of claim 1, wherein at least one pre-reforming chamber contains Ni catalyst and at least one pre-reforming chamber contains precious metal catalyst.

15: The process of claim 14, wherein said precious metal catalyst is selected from the group consisting of Pt, Pd, and Ru.

16: The process of claim 1, wherein each pre-reforming chamber is a stand alone reactor.

17: The process of claim 1, wherein at least two pre-reforming chambers are contained in a single vessel.

18: The process of claim 1, wherein said third pre-reformed stream is subsequently introduced into a steam methane reformer as feed.

19: The process of claim 1, wherein said third pre-reformed stream is subsequently introduced into an autothermal reformer as feed.

20: The process of claim 19, wherein inter-stage heating is performed by the flue gas of a process heater for preheating other process streams, wherein said inter-stage heating is an integral part of the autothermal reforming process.

21: The process of claim 1, wherein said first pre-reforming chamber has a first space velocity, said second pre-reforming chamber has a second space velocity, said third pre-reforming chamber has a third space velocity, and wherein said first space velocity is lower than said second space velocity or said third space velocity.
